(19) United States
(12) Patent Application Publication
(10) Pub. No.: US 2012/0316210 A1
(43) Pub. Date: Dec. 13, 2012

(54) TOPICAL ANTIPARASITIC FORMULATIONS

(75) Inventors: Nathan Anthony Logan Chubb, Richland, MI (US); Guy Francis de Rose, Kalamazoo, MI (US); Sunil Thomas Kumar Narishetty, Portage, MI (US)

(73) Assignee: PFIZER INC., New York, NY (US)

(21) Appl. No.: 13/522,339

(22) PCT Filed: Jan. 4, 2011
(86) PCT No.: PCT/IB11/50027

§ 371 Date: Jul. 16, 2012

Related U.S. Application Data
(60) Provisional application No. 61/299,384, filed on Jan. 29, 2010.

(51) Int. Cl. A61K 31/4174; A61P 33/14; A61P 33/00

(52) U.S. Cl. 514/396

(57) ABSTRACT

This invention recites topical formulations comprising demiditraz, fipronil, an acid modifier, at least one veterinarily acceptable carrier, and optionally, at least one antioxidant for treating a parasitic infection or infestation in animals.

### FIELD OF THE INVENTION

This invention relates to topical formulations comprising demiditraz and fipronil for treating parasitic infestations in animals by topically applying the inventive formulations to said animals. In particular, this invention provides for spot-on formulations comprising demiditraz and fipronil, an acid modifier, and at least one veterinarily acceptable liquid carrier, and optionally, at least one antioxidant. This invention also provides for a method of treating a parasitic infection or infestation in animals. The inventive stable and non-irritating topical formulations exhibit activity against parasites, particularly ectoparasites such as fleas and ticks.

### BACKGROUND

Parasitic diseases may be caused by either endoparasites or ectoparasites. As used herein endoparasites refer to those parasites living inside the body of the host, either within an organ (such as the stomach, lungs, heart, intestines, etc.) or simply under the skin. Ectoparasites are those parasites that live on the outer surface of the host but still draw nutrients from the host. Ectoparasites which infest animals include arthropods, such as ticks, fleas, mites, mosquitoes, lice, and the like and infections by these parasites can result in transmission of serious and even fatal diseases. Infestations by ectoparasitic arthropods including but not limited to ticks, mites, lice, stable flies, hornflies, blowflies, face flies, fleas, mosquitoes and the like are also a serious problem. Infection by these parasites results not only in loss of blood and skin lesions, but also can interfere with normal eating habits thus causing weight loss. Ectoparasitic infestations of a host can also result in transmission of serious diseases including but not limited to encephalitis, anaplasmosis, babesiosis, rocky mountain spotted fever, lyme disease, ehrlichiosis, West Nile virus, swine pox, malaria, yellow fever, and the like, many of which can be fatal to the host. Animals may be infected by several species of parasite(s) at the same time since infection by one parasite may weaken the animal and make it more susceptible to infection by a second species of parasite.

Therefore, there is a need for improved antiparasitic agents for use with animals, and in particular there is a need for improved insecticides and acaricides. Furthermore there is a need for improved topical products with convenient administration and which contain at least two antiparasitic agents which can be used to effectively treat ectoparasites, such as insects (e.g., fleas, lice, and flies) and acarids (e.g., mites and ticks). Such products would be particularly useful for the treatment of companion animals, such as cats, dogs, llamas, horses, and livestock, such as cattle, bison, swine, sheep, and goats.

The compounds currently available for insecticidal and acaricidal treatment of companion animals and livestock do not always demonstrate good activity, good speed of action, or a long duration of action. Most treatments contain hazardous chemicals that can have serious consequences, including lethality from accidental ingestion. Persons applying these agents are generally advised to limit their exposure. Pet collars and tags have been utilized to overcome some problems, but these are susceptible to chewing, ingestion, and subsequent toxicological effects to the animal. Thus, current treatments achieve varying degrees of success which depend partly on toxicity, method of administration, and efficacy. Currently, some agents are actually becoming ineffective due to parasitic resistance.

While it is known that it is sometimes possible to combine various parasiticides in order to broaden the antiparasitic spectrum, it is not always possible to predict whether these antiparasitics can be formulated to ensure a stable composition. For this reason, there is a need for an effective and stable antiparasitic formulation which may be easily administered, is tolerated, and will provide efficacy.

Various methods of formulating topical antiparasitic formulations are known in the art. U.S. Pat. No. 5,045,536 recites a large number of solvent systems for preparing formulations for localized topical application, some of which are known irritants. U.S. Pat. No. 6,395,765 recites a specific formulation wherein a crystallization inhibitor is required to ensure solubility and flowability. U.S. Pat. Nos. 6,482,425 and 6,426,333 recite fipronil combinations. However, neither discuss the complexities of actually preparing a stable combination. There is a need for a stable, effective, and less irritating topical formulation comprising at least two antiparasitic agents.

### SUMMARY

The present invention provides for topical formulations for the treatment of parasitic infestations in animals, and in particular, companion animals and livestock. The antiparasitic formulations of the present invention may be used to prevent, treat, repel, and control acarids and insect infection and infestation in animals. In addition, the invention contemplates the control and prevention of tick borne diseases, for example, Lyme disease, canine and bovine anaplasmosis, canine ehrlichiosis, canine rickettsiosis, canine and bovine babesiosis, epizootic bovine abortion, and theileriosis. Thus, according to the present invention, there is provided a spot-on formulation comprising a combination of effective amounts of demiditraz and fipronil.

In one aspect of the present invention is a topical formulation which comprises a parasitically effective amount of a) a veterinarily and parasiticidally effective amount of demiditraz, b) a veterinarily and parasiticidally effective amount of fipronil, c) an acid modifier, d) at least one veterinarily acceptable carrier, and optionally e) at least one antioxidant.

In another aspect of the instant invention, the acid modifier is a weak acid. In yet another aspect, the weak acid is a carboxylic acid derivative. In yet another aspect, the carboxylic acid derivative is a monocarboxylic, dicarboxylic, or tricarboxylic acid. Non-exclusive examples of a carboxylic acid derivative includes: acetic, caprylic, capric, oleic, lauric, myristic, stearic, linoleic, linolenic, tartaric, malic, succinic, adipic, azelaic, sebacic, citric acid, and the like, or mixtures thereof. In another aspect of the invention, the weak acid modifier is citric acid, adipic acid, lauric acid, or mixtures thereof.

In another aspect of the invention, the amount of the weak acid modifier in the topical formulation ranges from about 0.1 to about 1.5 milliequivalent (mEq) to the amount of demiditraz. In another aspect of the invention, the weak acid modifier ranges from about 0.2 to about 1.1 mEq to the amount of demiditraz. In yet another aspect of the invention, the weak acid modifier is about 0.2 mEq to about 0.8 mEq to the amount of demiditraz.

In another aspect of the invention, the acid modifier is a strong acid. In yet another aspect of the invention, the strong acid is a sulfonic acid derivative or an inorganic acid. Non-exclusive examples of sulfonic acid derivatives include: methane-sulfonic acid, ethane-sulfonic acid, p-toluene sulfonic acid, dodecylbenzene-sulfonic acid, and the like. In yet another aspect of the invention, the strong acid modifier is p-toluene sulfonic acid.

In yet another aspect of the invention, the amount of the strong acid modifier ranges from about 0.2 mEq to about 1.2 mEq to the amount of demiditraz. In another aspect of the invention, the strong acid modifier is in the range of about 0.3 mEq to about 1.0 mEq to the amount of demiditraz. In yet another aspect of the invention, the strong acid modifier is about 0.3 mEq to about 0.8 mEq to the amount of demiditraz.

In another aspect of the invention, the topical formulation comprises at least one veterinarily acceptable carrier, or a mixture thereof. The veterinarily acceptable carrier can provide numerous functions to the formulation, for example, solubility, stability, tolerability (e.g., anti-irritant), flowability, and the like. Non-exclusive examples of veterinarily acceptable carriers include: alcohol (e.g., methanol, ethanol, isopropyl (IPA), and the like), glycol ether, N-methyl pyrrolidinone (NMP), polyvinylpyrrolidinone (PVP), 2-pyrrolidone, gamma-hexylactone, methoxy propyl acetate (MPA), glycerol formal, glycerin, triacetin, d-panthenol, avenanthramides, water, and the like, or mixtures thereof.

In another aspect of the invention, the veterinarily acceptable carrier is selected from a di(C₂₋₄ glycol) mono(C₁₋₄ alkyl)ether, water, ethanol, NMP, PVP, MPA, triacetin, glycerin, gamma-hexylactone, glycerol formal, or mixtures thereof. In yet another aspect of the invention, the veterinarily acceptable carrier is NMP, MPA, PVP, diethylene glycol monomethyl ether (DEGMME), dipropylene glycol monomethyl ether (DPGMME), ethanol, water, or mixtures thereof.

In another aspect of the invention, the topical formulation further comprises at least one antioxidant. Non-exclusive examples of antioxidants include: vitamin C, vitamin E, propylgallate, 2-t-butyl-4-methoxyphenol (BHA), and 2,6-di-t-butyl-4-methylphenol (BHT), disodium EDTA, sodium sulfite, ascorbyl palmitate, and the like, or mixtures thereof. In another aspect of the invention, the antioxidant is selected from BHA, BHT, or a mixture thereof.

In another aspect of the invention is the use of a veterinary parasitical topical formulation comprising a) a veterinarily and parasiticidally effective amount of demiditraz, b) a veterinarily and parasiticidally effective amount of fipronil, c) an acid modifier; d) at least one veterinarily acceptable carrier; and optionally, e) at least one antioxidant for the treatment of a parasitic infestation or infection in animals. Non-exclusive examples of animals include companion animals, for example, cat, dog, and horse, and livestock, for example, cattle, swine, ovine, and caprine. In another aspect of the invention, the animal is selected from dog, cat, horse, cattle, and swine. In another aspect of the invention, the animal is a dog.

In another aspect of the invention is a veterinary parasitical topical formulation. The formulation can be administered topically as a spot-on, pour-on, multi-spot-on, stripe-on, comb-on, or roll-on formulation. In another aspect of the invention, the formulation is administered as a spot-on formulation.

In yet another aspect of the invention, is a method for treating an animal with a parasitic infection or infestation that includes the step of administering to said animal, in need of such treatment, a therapeutically effective amount of a formulation of the present invention.

Formulations of the present invention alone, or in combination with an additional third veterinary agent, may be administered as (a) a single veterinary composition which comprises a formulation of the present invention and at least one additional veterinary agent as described herein, and optionally, at least one veterinarily acceptable carrier; or (b) two separate veterinary compositions comprising (i) a first composition comprising a formulation of the present invention, and (ii) a second composition comprising at least one additional veterinary agent, as described herein and optionally, at least one veterinarily acceptable carrier. The combinatorial compositions optionally comprise at least one antioxidant. The veterinary compositions may be administered simultaneously or sequentially and in any order.

All of the recited US and European (EP) patents described herein are incorporated by reference.

### DEFINITIONS

For purposes of the present invention, as described and claimed herein, the following terms and phrases are defined as follows:

“About” when used in connection with a measurable numerical variable, refers to the indicated value of the variable and to all values of the variable that are within the experimental error of the indicated value (e.g., within the 95% confidence interval for the mean) or within 10 percent of the indicated value, whichever is greater.

“Acid modifier” as used herein, unless otherwise indicated, refers to a weak or strong acid capable of lowering the apparent pH of the formulation. The weak acids may also partially protonate demiditraz or other formulation carriers. The strong acids may wholly protonate demiditraz or other formulation carriers.

“Active ingredient(s)”, as used herein, unless otherwise indicated, refers to the compounds demiditraz and fipronil.

“Additional veterinary agent(s)” as used herein, unless otherwise indicated, refers to other veterinary compounds or products that provide a therapeutically effective amount of said agent(s) that are useful for the treatment of a parasitic infection or infestation in animals, as described herein.

“Animal” as used herein, unless otherwise indicated, refers to an individual animal. Animal includes both livestock and companion animals. Non-exclusive examples of livestock include swine, ovine, bovine, and caprine. Non-exclusive examples of companion animals include dogs, cats, and horses.

“Infestation”, as used herein, unless otherwise indicated, refers to the state or condition of having parasites on the body. Furthermore, the infestation may lead to an infection on or in the animal, which may be microbial, viral, or fungal.

“Parasite(s)”, as used herein, unless otherwise indicated, refers to ectoparasites. Ectoparasites are organisms of the Arthropoda phylum (arachnids and insects) which feed through or upon the skin of its host. Examples of arachnids are of the Order Acarina, for example, ticks and mites. Examples of parasitic insects are of the Order Siphonaptera and Phthiraptera, for example, fleas and biting and sucking lice.

“Therapeutically effective amount”, as used herein, unless otherwise indicated, refers to an amount of the compounds of the present invention that (i) treat or prevent the particular parasitic infection or infestation, and (ii) attenuates, ameliorates, or eliminates one or more symptoms of the particular parasitic infection or infestation, described herein. The therapeutically effective amount is both parasiticidally and veterinarily effective.

“Treatment”, “treating”, and the like, as used herein, unless otherwise indicated, refers to reversing, alleviating, or preventing the parasitic infection, infestation, or condition. As used herein, these terms also encompass, depending on the condition of the animal preventing the onset of a disorder or condition, or of symptoms associated with a disorder or condition, including reducing the severity of a disorder or condition or symptoms associated therewith prior to affliction with said infection or infestation. Thus, treatment can refer to administration of the formulation of the present invention to an animal that is not at the time of administration afflicted with the infection or infestation, for example, as prophylactic treatment. Treating also encompasses preventing the recurrence of an infection or infestation or of symptoms associated therewith as well as references to “control” (e.g., kill, repel, expel, incapacitate, deter, eliminate, alleviate, minimize, and eradicate).

“Veterinarily acceptable” as used herein, unless otherwise indicated, indicates that the substance or composition must be compatible chemically and/or toxicologically, with the other ingredients comprising a formulation, composition, and/or the animal being treated therewith.

### DETAILED DESCRIPTION OF THE INVENTION

It is to be understood by one of ordinary skill in the art that the present discussion is a description of exemplary embodiments only and is not intended as limiting the broader aspects of the present invention. In fact, it will be apparent to those skilled in the art that various modifications and variations can be made in the present invention without departing from the scope or spirit of the invention. For instance, features illustrated or described as part of one embodiment can be used in another embodiment to yield a still further embodiment. It is intended that the present invention cover such modifications and variations as come within the scope of the appended claims and their equivalents.

The present invention provides for a topical veterinary formulation for the treatment of a parasitic infection or infestation in animals which comprises a composition comprising (a) a veterinarily effective amount of demiditraz, with the formula:

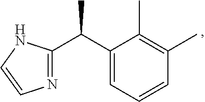

b) a veterinarily effective amount of fipronil, with the formula:

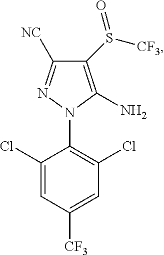

(c) an acid modifier, (d) at least one veterinarily acceptable carrier, and optionally e) at least one antioxidant.

The compounds fipronil and demiditraz can be synthesized by synthetic routes that include processes analogous to those well known in the chemical arts, and as detailed in U.S. Pat. No. 7,592,362 and European Patent No. EP0295117. The amounts of these compounds are easily determined by a skilled artisan and further depend on the dose amount and dose volume of the final formulation. Representative amounts of a veterinarily effective amount of demiditraz ranges from about 1 mg/kg to about 30 mg/kg, with a preferred range of about 10 mg/kg to about 25 mg/kg. The more preferred range of demiditraz is about 15 mg/kg to about 20 mg/kg. The most preferred amount for demiditraz is 15 mg/kg or 20 mg/kg. The preferred amount of demiditraz in the formulation ranges from about 100 mg/mL to about 200 mg/mL. More preferred, the amount of demiditraz is about 110 mg/mL to about 175 mg/mL. Even more preferred, the amount of demiditraz is about 112 mg/mL or about 150 mg/mL. Most preferred, the amount of demiditraz is 150 mg/mL.

A representative amount of a veterinarily effective amount of fipronil is about 1 mg/kg to about 12 mg/kg, with a preferred range of about 5 mg/kg to about 8 mg/kg. The most preferred amount of fipronil is 6.7 mg/kg. A preferred amount of fipronil ranges from about 25 mg/mL to about 75 mg/mL. More preferred, fipronil ranges from about 40 mg/mL to about 60 mg/mL. Even more preferred, the amount of fipronil is about 50 mg/mL. Dose volume for the final formulation ranges from about 0.100 mL/kg to about 0.150 mL/kg of animal body weight. More preferred, dose volume ranges from about 0.125 mL/kg to about 0.140 mL/kg of animal body weight. Even more preferred, dose volume is about 0.1333 mL/kg. Most preferred, dose volume is 0.1333 mL/kg of animal body weight.

A typical parasiticidal topical formulation can be prepared using conventional dissolution and mixing procedures, for example, in ‘Remington's Veterinary Sciences’, 19th Edition (Mack Publishing Company, 1995; and “Veterinary Dosage Forms: Tablets, Vol. 1”, by H. Lieberman and L. Lachman, Marcel Dekker, N.Y., 1980 (ISBN 0-8247-6918-X). These typical formulations generally contain a carrier including solvents, buffers, stabilizing agents, surfactants, wetting agents, lubricating agents, emulsifiers, suspending agents, preservatives, antioxidants, and the like. The particular carrier used will depend upon the means and purpose for which the compound of the present invention is being applied.

As described herein, the formulations of the instant invention may further comprise at least one antioxidant. Non-exclusive antioxidants include vitamin C (ascorbic acid), vitamin E (tocopherol), vitamin E derivatives, 2-t-butyl-4-methoxyphenol (BHA), 2,6-di-t-butyl-4-methylphenol (BHT), propyl gallate, and the like. The preferred amount of an antioxidant ranges from about 1 mg/mL to about 3 mg/mL, with a preferred amount of about 2 mg/mL.

When demiditraz and fipronil were formulated using conventional dissolution and mixing procedures, fipronil was shown to be unstable. Demiditraz is a weak base with a pKa of 7.2. Fipronil is also a weak base due to the presence of the imidazole ring. Titration experiments showed that fipronil degrades rapidly at pH levels above 7.5. An acid modifier (or mixtures thereof) was added to the formulation in a molar equivalent relative to the amount of demiditraz to adjust the pH below 7.5 and reduce or eliminate the nucleophilic reaction between fipronil and demiditraz. The acid modifier includes both weak and strong acids. The weak acids include sugar acids (glyceric, gluconic, ascorbic, tartaric, and the like), carboxylic acid derivatives (mono-, di-, and tri-chloro acetic acid, and mono-, di-, and tri-fluoro acetic acid, benzoic, and the like) and mono-, di-, and tri-carboxylic acids. Non-exclusive examples of monocarboxylic acids include formic, acetic, propionic, butyric, oleic, valeric, caproic, enanthic, caprylic, linoleic, chloroacetic, dichloroacetic, oxalic, benzoic, pelargonic, capric, lauric, myristic, palmitic, stearic, and arachidic acid. Non-exclusive examples of di-carboxylic acids include oxalic, malonic, succinic, fumaric, aldaric, tartaric, glutaric, adipic, maleic, malic, pimelic, suberic, azelaic, sebacic, phthalic, isophtalic, and terephthalic acid. Non-exclusive examples of tri-carboxylic acids include citric, isocitric, aconitic, propane-1,2,3-tricarboxylic, and trimesic acid.

The addition of the weak acid in the formulation lowered the apparent pH of the formulation to about 6.5 to 7.5, however, fipronil degraded initially by about 5-6% followed by slower degradation to about 10-15% during an accelerated (70°) kinetic study. Strong acids (pka≦2) were added to the formulation to both lower apparent pH to about 2.5 to about 3.5 and to protonate demiditraz to prevent it from potentially acting as a nucleophile against fipronil. Non-exclusive examples of strong acids include inorganic acids (e.g., HCl, HBr, HI, HF, nitric acid, phosphoric acid, boric acid, sulfuric acid, and the like). Other strong acids include sulfonic acid and sulfonic acid derivatives thereof. Non-exclusive examples of sulfonic acid derivatives include methane-, ethane-, p-toluenesulfonic (p-TSA), and dodecylbenzenesulfonic acid. The use of a strong acid, e.g., p-TSA, increased fipronil stability, however, degradation was still about 5% after 12 weeks at 50° C.

In an effort to reduce or eliminate fipronil instability, particularly the initial degradation, different veterinary carriers were assessed. Non-exclusive examples of suitable veterinarily acceptable carriers include: alcohol (e.g., methanol, ethanol, propanol, isopropyl, propylene glycol, benzyl, and the like), water, glycol ether, N-methyl pyrrolidinone (NMP), 2-pyrrolidone, polyvinylpyrrolidinone (PVP), gamma-hexylactone, methoxypropyl acetate (MPA), triacetin, glycerin, glycerol formal, tetraglycol, isopropyl myristate, and mixtures thereof. Non-exclusive examples of glycol ether include ethylene glycol monomethyl ether, ethylene glycol monomethyl ether acetate, ethylene glycol monoethyl ether, ethylene glycol monoethyl ether acetate, ethylene glycol monopropyl ether, ethylene glycol monobutyl ether, ethylene glycol dimethyl ether, ethylene glycol diethyl ether, diethylene glycol, diethylene glycol monomethyl ether (DEGMME), diethylene glycol monoethyl ether (DEGMEE), diethylene glycol monomethyl ether ethanol, diethylene glycol monobutyl ether ethanol, diethylene glycol dimethyl ether, triethylene glycol diethyl ether, propylene glycol monomethyl ether, propylene glycol monomethyl ether acetate, dipropylene glycol, dipropylene glycol monomethyl ether (DPGMME), and the like. In addition, non-exclusive veterinarily acceptable carriers which are known to impart dermal tolerability can also be used as a carrier and include: d-panthenol, avenanthramides, bisabolol, alpha-lipoic acid, allantoin, sorbitol, potassium gluconate, lanolin, peramides, or mixtures thereof, and the like.

The initial instability of fipronil was reduced to about ≦1% during an accelerated (70° C.) 1-week stability study for DPGMME with a strong acid and for NMP with a strong or weak acid. During the same study, fipronil was shown to degrade by about 3 to 6% for weak acids in 2-pyrrolidone and weak acids in DPGMME.

When using a strong acid, the free base of demiditraz converted to the protonated form in-situ. The protonated form possesses different physiochemical properties than the freebase and may affect efficacy and/or safety. In an effort to assess equipotency of an in-situ salt to the free base of demiditraz (150 mg/mL), an efficacy study was conducted to compare the demiditraz salts of a low, medium, and high log P counterion of weak and strong acids versus the free base in DPGMME. For weak acid (1.1 mEq) formulations, the formulation was prepared as follows: 1) dissolve/mix weak acid in DPGMME to 50% final volume, 2) warm to 50° C., 3) add demiditraz and BHA, 4) warm to 50° C., 5) allow demiditraz to dissolve and cool to room temperature, and 6) q.s. with DPGMME. For strong acid (1.0 M equivalents) formulations, the formulation was prepared as follows: 1) dissolve/mix strong acid in DPGMME, 2) add acid solution to demiditraz and BHA to 75% total volume, 3) warm to 50° C., 4) once dissolved, add acid solution dropwise to achieve pH of 2.5 to 3.5, and 5) q.s. with DPGMME. The study was run for 5 weeks with tick challenge at Day −2, 7, 14, 21, 28 and 35. Efficacy was determined at Day 0, 9, 16, 23, 30 and 37 (Table 1).

TABLE 1
Efficacy results.

| Acid | Day 2 | Day 9 | Day 16 | Day 23 | Day 30 | Day 37 |
|---|---|---|---|---|---|---|
| Free Base | 100.0% | 100.0% | 100.0% | 100% | 99.2% | 97.8% |
| Adipic | 93.3% | 97.4% | 100.0% | 99.5% | 98.2% | 97.6% |
| Lauric | 99.6% | 100.0% | 100.0% | 100% | 100% | 98.3% |
| Oleic | 98.7% | 100.0% | 100.0% | 99.5% | 98.6% | 98.8% |
| Ethane-sulfonic | 97.4% | 98.2% | 100% | 100% | 100% | 99.4% |
| pTSA | 94.4% | 100.0% | 99.4% | 99.5% | 99.2% | 98.5% |
| dodecyl- | 96.7% | 100.0% | 97.8% | 99.2% | 98.2% | 100% |
| benzene- | | | | | | |
| sulfonic acid. | | | | | | |

All groups tested showed equivalent potency and persistence to demiditraz free base. The medium log P acids were also shown to be less irritating to the animals. Overall, the weak and strong acid formulations in DPGMME provided efficacy out through 37 days.

Tolerability of numerous formulations was assessed by topically applying a test formulation to the skin of a rabbit. Erythema, eschar formation, and edema was quantitated macroscopically using a scoring system that ranged from 0 to 4 (Table 2).

TABLE 2
Macroscopic Scoring for Rabbit Toleration

| Erythema and Eschar | | |
|---|---|---|
| Formation | Edema Formation | Score |
| None | None | 0 |
| Very Slight (barely | Very Slight (barely perceptible) | 1 |
| perceptible) | | |
| Well defined | Slight edema (edges of area well | 2 |
| | defined by definite raising) | |
| Moderate to severe | Moderate edema (raised ~1 mm) | 3 |
| Severe (beet redness) | Severe edema (raised >1 mm and | 4 |
| to slight eschar formation | extending beyond area of exposure) | |

Additionally, epidermal necrosis was assessed microscopically and was quantitated with a grading system. The following grade scores were: Grade 1 (minimal), the amount of change present barely exceeds that which is considered to be within normal limits (very few, rare, or very small); Grade 2 (trace), in general, the lesion is identifiable but of limited severity and no functional impairment (few or small size); Grade 3 (mild), the lesion is easily identified and minimal functional impairment is possible (moderate number or moderate size); Grade 4 (moderate), the lesion is prominent but there is significant potential for increased severity; and Grade 5 (severe), the degree of change is either as complete as considered possible or great enough in intensity or extent to expect significant tissue or organ dysfunction (extensive number or extensive size).

For the toleration studies, four to eight male New Zealand White Hra:(NZW)SPF rabbits, aged 5-6 months, were dosed per formulation. On Day −1, the hair was carefully clipped from the test site to avoid abrasion. A single formulation (0.15 mL/kg) was administered topically to the test site on Day 1. Each formulation contained 150 mg/mL demiditraz and 50 mg/mL fipronil, except where specified. Animals were observed twice daily for signs of general health. The test site was evaluated for erythema and edema at 2, 4, 6, 8, 24, and 48 hours post dose. Animals were necropsied following evaluation of skin reaction approximately 48 hours postdose. All animals were euthanized and skin samples were collected. Macroscopic and microscopic evaluations were conducted according to the scoring values described herein. (Table 3).

TABLE 3
Rabbit Tolerabiltiy

| | | | Epidermal |
|---|---|---|---|
| | Erythema | Edema | necrosis |
| Acid Modifier and Carrier | (animals/score) | (animals/score) | (animals/score) |
| 1.1 mEq lauric acid in DPGMME | 5/0, 3/1 | 8/0 | 2/0, 2/1, 2/2, |
| | | | 2/3 |
| 0.8 mEq adipic acid in DPGMME | 6/0, 1/1, 1/3 | 8/0 | 4/0, 1/1, 1/2, |
| | | | 1/3, 1/5 |
| 0.3 mEq citric acid in DPGMME | 1/0, 2/1, 1/2 | 4/0 | 1/1, 2/2, 1/3 |
| 0.3 mEq HCl acid in DPGMME | 3/0, 1/1 | 4/0 | 1/0, 1/1 1/2, |
| | | | 1/3 |
| 0.8 mEq citric acid in NMP | 4/0 | 4/0 | 3/0, 1/2 |
| 0.8 mEq HCL in NMP | 2/0, 1/1, 1/2 | 4/0 | 2/1, 1/3, 1/4 |
| 0.8 mEq lauric acid in γ-hexalactone | 1/0, 3/1 | 4/0 | 2/0, 1/1, 1/2 |
| 0.8 mEq lauric acid in γ-hexalactone/NMP | 2/0, 2/1 | 4/0 | 1/1, 1/2, 1/3, |
| (70:30) | | | 1/4 |
| 0.3 mEq citric acid in γ-hexalactone/NMP | 2/0, 2/3 | 2/0, 1/1, 1/2 | 3/0, 1/5 |
| (70:30) | | | |
| 0.8 mEq lauric acid in MPA/NMP (70/30) | 4/0 | 4/0 | 2/0, 2/1 |
| 0.3 mEq citric acid in MPA/NMP (70/30) | 4/0 | 4/0 | 3/0, 1/1 |
| 0.8 mEq lauric acid in triacetin/NMP | 4/0 | 4/0 | 1/0, 1/1, 2/4 |
| (60/40) | | | |
| 0.4 mEq citric acid in triacetin/NMP | 4/0 | 4/0 | 3/0, 1/1 |
| (60/40) | | | |
| 0.3 mEq HCL in γ-hexalactone | 3/0, 1/1 | 4/0 | 3/0, 1/4 |
| 0.5 mEq HCL in γ-hexalactone/NMP | 4/0 | 4/0 | 3/0, 1/2 |
| (70/30) | | | |
| 0.8 mEq lauric acid in NMP + glycerin | 4/0 | 4/0 | 4/4 |
| (20%) | | | |
| 0.4 mEq citric acid in NMP + glycerin | 4/0 | 4/0 | 3/0, 1/1 |
| (20%) | | | |
| 0.8 mEq lauric acid in NMP + PVP (10%) | 2/0, 2/1 | 4/0 | 4/4 |
| 0.8 mEq citric acid in NMP + PVP (10%) | 3/0, 1/1 | 4/0 | 4/0 |
| 0.8 mEq adipic acid in NMP + bisabolol | 4/1 | 4/0 | 1/0, 2/2, 1/3 |
| (0.2%) | | | |
| 0.8 mEq adipic acid in NMP + | 1/0, 3/1 | 4/0 | 2/0, 1/1, 1/3 |
| aventhramides (2%) | | | |
| 1.0 mEq ascorbic acid in NMP | | 4/0 | 3/0, 1/2 |
| 0.4 mEq adipic acid in DEGMEE | 3/0, 1/1 | 4/0 | 2/0, 1/1, 1/2 |
| 0.4 mEq adipic acid in DEGMEE + PVP | 2/0, 2/1 | 4/0 | 4/0 |
| 0.8 mEq adipic acid in NMP/IPA (70/30) | 4/0 | 4/0 | 1/0, 2/1, 1/2 |
| 1.1 mEq lauric acid in DEGMME + PVP | 4/0 | 4/0 | 2/0, 1/3, 1,4 |
| 0.8 mEq lauric acid in DEGMME + glycerin | 4/0 | 4/0 | 3/0, ½ |
| 0.2 mEq citric acid in glycerol formal | 4/0 | 4/0 | 4/0 |
| 0.4 mEq citric acid in NMP/water (75/25) | 4/0 | 4/0 | 4/0 |
| 0.8 mEq lauric + 0.7 mEq citric acid in NMP | 4/0 | 4/0 | 1/0, 2/1, 1/3 |
| 0.8 mEq lauric acid in NMP* | 5/0, 3/1 | 8/0 | 1/0, 1/1, 1/2, |
| | | | 2/3 |
| 0.8 mEq citric acid in NMP + | 3/0, 1/1 | 4/0 | 2/0, 1/1, 1/4 |
| avenanthramides | | | |
| 0.4 mEq citric acid in NMP + d-panthenol | 3/0, 1/1 | 4/0 | 2/0, 1/1, 1/4 |
| 0.2 mEq citric acid in NMP + IPA | 3/0, 1/1 | 4/0 | 2/0, 2/2 |

*Demiditraz at 200 mg/mL and fipronil at 67 mg/mL.

The formulation of the present invention is envisioned to be administered topically to an animal at least once every 4 to 6 weeks, depending upon the parasite involved. The formulations of the present invention may be administered topically to the skin or mucosa, that is dermally or transdermally, to an animal. Topical applications include spot-on, pour-on, multi-spot-on, stripe-on, comb-on, or roll-on formulations.

The formulation of the instant invention may be administered alone or in combination with at least one additional veterinary agent. Non-exclusive examples of other topical veterinary agents include: amitraz, DEET, insect growth regulators (e.g., hydroprene, kinoprene, methoprene, pyriproxyfen, and the like), permethrin, pyrethrins, spinosad, and the like).

The formulations of the present invention are useful as parasiticides for the treatment of parasitic infections or infestations in an animal. The formulations of the present invention have utility as a parasiticide, in particular, as an acaricide and insecticide. They may, in particular, be used in the fields of veterinary medicine, livestock husbandry and the maintenance of public health: against acarids and insects which are parasitic upon vertebrates, particularly warm-blooded vertebrates, including domestic animals such as dogs, cats, cattle, sheep, goats, horses, llamas, bison, and swine. Some non-limiting examples of acaride and insect parasites include: ticks (e.g., Ixodes spp., Rhipicephalus spp., Boophilus spp., Amblyomma spp., Hyalomma spp., Haemaphysalis spp., Dermacentor spp., Ornithodorus spp., and the like); mites (e.g., Dermanyssus spp., Sarcoptes spp., Psoroptes spp., Chorioptes spp., Demodex spp., and the like); chewing and sucking lice (e.g., Damalinia spp., Linognathus spp., and the like); fleas (e.g., Siphonaptera spp., Ctenocephalides spp., and the like); and biting flies and midges (e.g., Tabanidae spp., Haematobia spp., Stomoxys spp., Dermatobia spp., Simuliidae spp., Ceratopogonidae spp., Psychodidae spp., and the like).

The formulations of the present invention are of particular value in the control of ectoparasites and insects which are injurious to, or spread or act as vectors of diseases in companion and livestock animals, for example those hereinbefore mentioned, and more especially in the control of ticks, mites, lice, fleas, midges and biting, nuisance and myiasis flies. They are particularly useful in controlling acarids and insects which feed on the skin or suck the blood of the animal, for which purpose they may be administered topically.

Any of the formulations of the present invention may be administered directly to the animal subject and/or indirectly by applying it to the local environment in which the animal dwells (such as bedding, enclosures, and the like). Direct administration includes contacting the skin or fur of a subject animal with the active compounds.

The formulations of the present invention have value for the treatment and control of the various lifecycle stages of insects and parasites including egg, nymph, larvae, juvenile and adult stages.

The present invention also relates to a method of administering a formulation of the present invention to animals in good health comprising the application to said animal to reduce or eliminate the potential for human parasitic infection or infestation from parasites carried by the animal and to improve the environment in which the animals and humans inhabit.

### Examples

The following formulation examples (Table 4) are construed to be non-exclusive. In the examples, the amount of demiditraz is 150 mg/mL (15% w/v) and fipronil is 50 mg/mL (5% w/v). The formulations may also comprise at least one anti-oxidant ranging in concentration from about 1 mg/mL (0.1% w/v) to about 3 mg/mL (0.3% w/v). The formulation can be prepared by adding and dissolving the acid modifier to the carrier (i.e. NMP, glycerol formal). Demiditraz, and optionally, an antioxidant can be added to the carrier-acid modifier solution and dissolved. Fipronil can then be added and dissolved. Additional carriers (i.e., glycerin, PVP) can be added based on a w/v %. The final volume is brought to about 1 mL with the carrier.

TABLE 4
Formulation Examples

| # | Acid Modifier | Carrier |
|---|---|---|
| 1 | 0.8 mEq lauric acid | MPA/NMP (70/30) (q.s.) |
| 2 | 0.4 mEq citric acid | glycerin (20% w/v) + NMP (q.s.) |
| 3 | 0.8 mEq citric acid | NMP (q.s.) |
| 4 | 0.8 mEq citric acid | PVP (10% w/v) + NMP (q.s.) |
| 5 | 0.2 mEq citric acid | Glycerol formal (q.s.) |
| 6 | 0.4 mEq citric acid | Triacetin/NMP (60/40) (q.s.) |
| 7 | 0.4 mEq citric acid | NMP/Water (75/25) (q.s.) |
| 8 | 0.3 mEq citric acid | MPA/NMP (70/30) (q.s.) |
| 9 | 0.8 mEq lauric acid | Gamma-hexalactone (q.s.) |

1. A topical formulation for the treatment of a parasitic infestation in an animal, which comprises
a) a therapeutically effective amount of demiditraz;
b) a therapeutically effective amount of fipronil;
c) an acid modifier;
d) at least one veterinarily acceptable carrier; and optionally,
e) at least one antioxidant.

2. The formulation of claim 1, wherein the acid modifier is a weak acid or an inorganic acid.

3. The formulation of claim 2 wherein the weak acid is a carboxylic acid derivative and the inorganic acid is hydrochloric acid.

4. The formulation of claim 3 wherein the carboxylic acid derivative is selected from adipic, citric, and lauric acid.

5. The formulation of claim 2 wherein the veterinarily acceptable carrier is selected from an alcohol, N-methyl pyrrolidinone, 2-pyrrolidone, polyvinylpyrrolidinone, triacetin, gamma-hexylactone, glycerin, glycerol formal, methoxypropyl acetate, water, and a glycol ether, or a mixture thereof.

6. The formulation of claim 2, further comprising at least one antioxidant.

7. The formulation of claim 6 wherein the antioxidant is selected from Vitamin C, Vitamin E, propylgallate, 2-t-butyl-4-methoxyphenol, 2,6-di-t-butyl-4-methylphenol, disodium EDTA, sodium sulfite, and ascorbyl palmitate, or a mixture thereof.

8. A topical formulation for the treatment of a parasitic infestation in an animal, which comprises
a) a therapeutically effective amount of demiditraz;
b) a therapeutically effective amount of fipronil;
c) an acid modifier selected from lauric acid, citric acid, adipic acid, or hydrochloric acid; and
d) a veterinarily acceptable carrier selected from N-methyl pyrrolidinone, methoxypropyl acetate, triacetin, gamma-hexylactone, glycerin, ethanol, polyvinylpyrrolidinone, a glycol ether, or a mixture thereof; and optionally
e) at least one antioxidant.

9. The formulation of claim 8, wherein the topical formulation comprises about 150 mg/mL demiditraz, about 50 mg/mL fipronil, and the acid modifier is about 0.2 mEq to about 1.2 mEq to the amount of demiditraz.

10. The formulation of claim 9, wherein the topical formulation further comprises at least one antioxidant.

11. The formulation of claim 1, wherein the formulation is administered topically to an animal as a spot-on, pour-on, multi-spot-on, stripe-on, comb-on, or roll-on formulation.

12. Use of a topical formulation which comprises
a) a therapeutically effective amount of demiditraz;
b) a therapeutically effective amount of fipronil;
c) an acid modifier;
d) at least one veterinary acceptable carrier; and optionally
e) at least one antioxidant,
for the treatment of a parasitic infestation in an animal.

13. The use of claim 12, wherein the animal is a companion animal or livestock.

14. The use of claim 13, wherein the companion animal is a dog, cat, or horse and livestock is bovine, swine, ovine, or caprine.

15. (canceled)

16. A method of treating a parasitic infection on an animal comprising topically administering an antiparasitic formulation comprising
a) a therapeutically effective amount of demiditraz;
b) a therapeutically effective amount of fipronil;
c) an acid modifier;
d) at least one veterinary acceptable carrier; and optionally
e) at least one antioxidant,
to an animal in need thereof.

17. The method of claim 16 wherein the parasitic infection is an ectoparasitic infection.

18. The method of claim 16 wherein the acid modifier is a weak acid or an inorganic acid.

19. The method of claim 16 wherein the weak acid is a carboxylic acid derivative and the inorganic acid is hydrochloric acid.

20. The method of claim 19 wherein the carboxylic acid derivative is selected from adipic, citric, and lauric acid.

21. The method of claim 19 wherein the veterinarily acceptable carrier is selected from an alcohol, N-methyl pyrrolidinone, 2-pyrrolidone, ethanol, polyvinylpyrrolidinone, triacetin, gamma-hexalactone, glycerin, glycerol formal, methoxypropyl acetate, water, and a glycol ether, or a mixture thereof, and further comprising at least one antioxidant.
